(19) United States
(12) Patent Application Publication
(10) Pub. No.: US 2021/0290127 A1
(43) Pub. Date: Sep. 23, 2021

(54) Information Processing Device and Method

(71) Applicant: Hitachi, Ltd., Tokyo (JP)

(72) Inventors: Akiko OBATA, Tokyo (JP); Tsukasa FUNANE, Tokyo (JP)

(21) Appl. No.: 17/201,858

(22) Filed: Mar. 15, 2021

(30) Foreign Application Priority Data
Mar. 19, 2020 (JP) 2020-050191

(51) Int. Cl. A61B 5/16; G09B 5/06; G09B 19/00; A61B 5/00

(52) U.S. Cl. CPC A61B 5/16; A61B 5/4803; G09B 19/00; G09B 5/06

(57) ABSTRACT

An information processing device and an information processing method of the information processing device to optimize information presented by an information presenter to an information recipient determine a knowledge level and an understanding level of the information recipient with respect to the information based on a biological activity of the information recipient acquired by a sensor, and determine a feature of the information presented by the information presenter to the information recipient, and convert a presentation format of the information into a presentation format according to the knowledge level and the understanding level of the information recipient based on the determined knowledge level and the determined understanding level of the information recipient with respect to the information and the determined feature of the information.

### BACKGROUND OF THE INVENTION

### 1. Field of the Invention

The present invention relates to an information processing device and a method, and is suitable for being applied to, for example, an information presentation format optimization device that optimizes a presentation format of information presented to an information recipient by an information presenter for the information recipient.

### 2. Description of the Related Art

Conventionally, a method has been proposed in which a degree of understanding on information of an information recipient who receives a presentation of the information from an information presenter is estimated and evaluated, and the information according to the estimated and evaluated degree of understanding is presented to the information recipient. For example, in JP 2019-148919 A, the degree of understanding of a user for an event is acquired based on a user's utterance content and a user's answer to a question, and response information containing scenario information according to the acquired degree of understanding is transmitted to the user.

### SUMMARY OF THE INVENTION

However, the invention disclosed in JP 2019-148919 A only discloses that uniform scenario information according to the degree of understanding of the user is provided, and there is no disclosure or suggestion as to a specific presentation format for presenting the scenario information.

The invention has been made in consideration of the above points, and is to provide an information processing device and a method which can prevent misunderstandings and lack of understanding of an information recipient with respect to information by presenting the information in an optimal presentation format that is easy for the information recipient to understand.

In order to solve such a problem, in the invention, an information processing device for optimizing information presented by an information presenter to an information recipient includes an attribute determination unit that determines a knowledge level and an understanding level of the information recipient with respect to the information based on biological activity information of the information recipient acquired by a sensor, a presentation information feature determination unit that determines a feature of the information presented by the information presenter to the information recipient, and a feedback optimization unit that converts a presentation format of the information into a presentation format according to the knowledge level and the understanding level of the information recipient based on the knowledge level and the understanding level of the information recipient with respect to the information determined by the attribute determination unit and the feature of the information determined by the presentation information feature determination unit.

Further, in the invention, an information processing method for optimizing information presented by an information presenter to an information recipient using an information processing device includes a first step in which a knowledge level and an understanding level of the information recipient with respect to the information are determined based on a biological activity of the information recipient acquired by a sensor, and a feature of the information presented by the information presenter to the information recipient is determined, and a second step in which a presentation format of the information is converted into a presentation format according to the knowledge level and the understanding level of the information recipient based on the determined knowledge level and the determined understanding level of the information recipient with respect to the information and the feature of the information.

According to the information processing device and the method of the invention, the display format of the information presented by the information presenter to the information recipient can be converted into an optimum presentation format in which the information recipient can easily understand the information and presented.

According to the invention, it is possible to realize an information processing device and a method capable of effectively preventing misrecognition and lack of understanding of the information recipient for information.

### BRIEF DESCRIPTION OF THE DRAWINGS

FIG. 1 is a block diagram illustrating an overall configuration of an information presentation format optimization system according to this embodiment;

FIG. 2 is a diagram provided for explaining the features extracted from the information by a presentation information feature determination program;

FIG. 3 is a diagram provided for explaining the feature amount about a biological activity acquired from a sensor output by a biological activity analysis engine;

FIG. 4 is a diagram illustrating a configuration example of a knowledge level determination database;

FIG. 5 is a diagram illustrating a configuration example of an understanding level determining database;

FIG. 6 is a diagram illustrating a configuration example of a visual recognition level determining database;

FIG. 7 is a diagram illustrating a configuration example of a cognitive conflict level determining database;

FIG. 8 is a diagram illustrating a configuration example of a voice speed level determining database;

FIG. 9 is a diagram illustrating a configuration example of a voice intonation level determining database;

FIG. 10 is a diagram illustrating a configuration example of a comprehensive determining optimization database;

FIG. 11 is a diagram illustrating a configuration example of a conversion optimization database;

FIG. 12 is a flowchart illustrating a processing procedure of the information presentation format optimization program;

FIG. 13 is a flowchart illustrating a processing procedure of an attribute determination program;

FIG. 14 is a flowchart illustrating a processing procedure of the presentation information feature determination program; and

FIG. 15 is a flowchart illustrating a processing procedure of a prediction model creation program.

### DESCRIPTION OF THE PREFERRED EMBODIMENTS

Hereinafter, embodiments of the invention will be described in detail with reference to the drawings.

The embodiments described below are examples for explaining the invention, and are appropriately omitted and simplified for the sake of clarification of the description. The invention can be implemented in other various forms. Unless otherwise limited, each component may be singular or plural.

The position, size, shape, range, and the like of each component illustrated in the drawings may not necessarily represent the actual position, size, shape, range, and the like, in order to facilitate understanding of the invention. For this reason, the invention is not necessarily limited to the position, size, shape, range, and the like disclosed in the drawings.

As examples of various types of information, the expressions such as “table”, “list”, “queue”, and the like may be used. However, various types of information may be expressed by a data structure other than these. For example, various types of information such as “XX table”, “XX list”, “XX queue”, and the like may be “XX information”. In describing the identification information, expressions such as “identification information”, “identifier”, “name”, “ID”, and “number” are used, but these can be replaced with each other.

When there are a plurality of components having the same or similar functions, different subscripts may be given for the same reference numerals for explanation. In addition, when there is no need to distinguish between these components, the description may be omitted with subscripts omitted.

In the embodiment, a process performed by executing a program may be described. Here, a computer executes a program by a processor (for example, CPU, GPU), and performs a process defined by the program while using a storage resource (for example, memory), an interface device (for example, a communication port), and the like. Therefore, the subject of the processing performed by executing the program may be the processor.

Similarly, the subject of the process performed by executing the program may be a controller, an apparatus, a system, a computer, or a node, which have a processor. The subject of the process performed by executing the program may be an arithmetic unit, and may include a dedicated circuit for performing a specific process. Here, the dedicated circuit is, for example, a Field Programmable Gate Array (FPGA), an Application Specific Integrated Circuit (ASIC), a Complex Programmable Logic Device (CPLD), or the like.

The program may be installed in the computer from a program source. The program source may be, for example, a program distribution server or a computer-readable storage medium. In a case where the program source is a program distribution server, the program distribution server includes a processor and a storage resource for storing the program to be distributed, and the processor of the program distribution server may distribute the program to be distributed to another computer. In addition, in the following embodiments, two or more programs may be expressed as one program, or one program may be expressed as two or more programs.

### (1) Configuration of Information Presentation Format Evaluation System According to this Embodiment

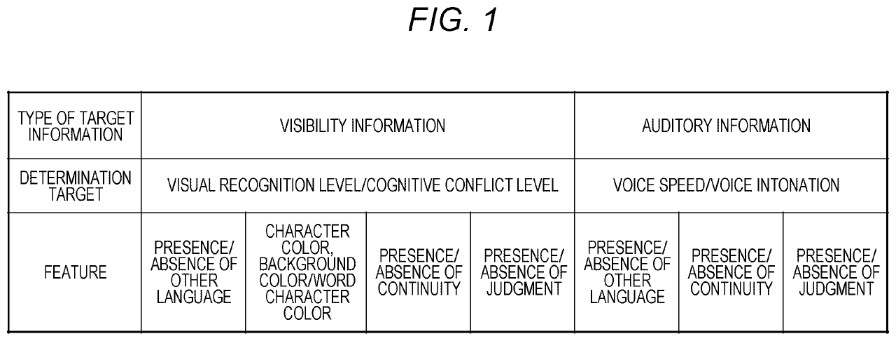

In FIG. 1, an information presentation format evaluation system 1 according to this embodiment is illustrated as a whole. This information presentation format evaluation system 1 is, for example, an information processing system used when a senior worker (information presenter) educates a plurality of new workers (information recipients) on work procedures in a factory, and includes a plurality of sensors 2 and an information presentation format optimization device 3.

The sensor 2 is an element or device that converts information about human biological activity into an electric signal, and is configured by camera, brain wave meter, cerebral blood flow measuring device, electrocardiogram, thermography, heart rate monitor, sphygmomanometer, readable pulse measuring device, or the like. The sensor 2 outputs the electric signal obtained by such conversion to the information presentation format optimization device 3.

The information presentation format optimization device 3 is configured by a general-purpose computer device which is provided with information processing resources such as a Central Processing Unit (CPU) 10, a memory 11, a storage device 12, and an output device 13.

The CPU 10 is a processor that controls the operation of the entire information presentation format optimization device 3. The memory 11 is configured by, for example, a non-volatile semiconductor memory and is used as a work memory of the CPU 10. A presentation information feature determination program 20, an attribute determination program 21, a feedback optimization program 22, and a prediction model creation program 25, which will be described later, are also stored and held in the memory 11.

The storage device 12 is configured by a non-volatile large-capacity storage device such as a hard disk device or a Solid State Drive (SSD), and stores various programs and data to be stored for a long period of time. A determination database group 23 and an optimization database group 24, which will be described later, are also stored and held in the storage device 12. The output device 13 is configured by, for example, a display device such as a liquid crystal panel or an organic Electro Luminescence (EL) panel, an acoustic device such as a speaker, and various devices capable of presenting information such as a printer to a user.

### (2) Information Presentation Format Optimization Function

Next, the information presentation format optimization function mounted on the information presentation format optimization device 3 will be described. This information presentation format optimization function is a function of evaluating the presentation format of information to be presented by an information presenter to an information recipient and attributes (knowledge level and understanding level) of the information recipient with respect to the information, and converting the presentation format of the information to a presentation formation according to the attributes of the information recipient based on the evaluation result and outputting the presentation format. As a result, it is possible to effectively prevent misrecognition and lack of understanding, and to shorten the time required to understand information.

Here, the “presentation format of information” in this embodiment indicates “color combination” and “word character color” in a case where the information is visually presented, such as characters, sentences, or figures (hereinafter, appropriately referred to as visibility information).

For example, with regard to the “color combination”, it has been confirmed through research and experiments that setting the characters to yellow and the background color to black easily attracts human attention and the discrimination error is reduced. Also, in the case of a sentence that instructs to perform two or more tasks, processes, or operations in succession, such as “perform work A and then stop process of work B”, it is also known that a visual recognition level is improved by setting the color combination of “perform” and “stop” to a combination that lowers the cognitive load (for example, “perform” is blue, and “stop” is red).

In the case of visual information (hereinafter, referred to as visibility information) in this way, the degree at which the visibility information can be visually recognized at a glance (hereinafter, referred to as a visual recognition level) changes depending on the color combination. Therefore, the visual recognition level can be improved by changing the color combination. By improving the visual recognition level of the visibility information, it is possible to suppress the judgment mistakes of the information recipient and reduce the frequency of occurrence of judgment mistakes.

Also, regarding “word character color”, it is confirmed through research and experiments that the cognitive load is increased in the case of mismatching of the meaning of the word and the character color such as notating the word “yellow” with red, or notating the word “stop” with blue or yellow.

In this way, in the case of visibility information, the degree of cognitive conflict with the visibility information (conflict with the rules and concepts acquired on a daily basis) is changed according to the notation color of the word (hereinafter, referred to as the word character color). Therefore, the cognitive conflict level can be reduced by changing the word character color. By reducing the cognitive conflict level of information, it is possible to suppress the judgment mistakes of the information recipient and reduce the frequency of occurrence of judgment mistakes.

Therefore, the information presentation format optimization device 3 of this embodiment determines the attribute of the information recipient based on the output of each sensor 2 when the information presented by the information presenter to the information recipient is visibility information. At the same time, the visual recognition level and cognitive conflict level of the visibility information are determined. Further, the information presentation format optimization device 3 evaluates the appropriateness of the current presentation format of the visibility information to the information recipient based on these determination results, and based on the evaluation result. The “color combination” and the “word character color” are outputted after being converted into visibility information of “color combination” and “word character color” according to the attribute of the information recipient.

On the other hand, when the information presented to the information recipient is information that is presented audibly, such as voice (hereinafter, appropriately referred to as auditory information), the “presentation format of information” indicates the “speed of voice (voice speed)” and the “intonation of voice (voice intonation)”.

For example, regarding the “voice speed”, when two or more pieces of information or words are presented by voice in succession, and the presentation interval of these information or words is less than 0.3 seconds, it is confirmed through experiments that it is subjectively difficult to judge by comparing the last presented information or word and the information or word presented immediately before the last one. In addition, when the presentation interval of information or words is less than 1.5 seconds, it is also confirmed that it feels subjectively difficult to judge by comparing the last presented information or word with the information or word presented last but two. Further, regarding “intonation”, it is known that the degree of understanding of the information recipient when the auditory information is presented by voice is higher when the intonation of phonology is extended.

In this way, in the case of auditory information, the degree of understandability of the auditory information received by the information recipient changes depending on the degree of information and word interval when information is presented by voice (hereinafter, referred to as voice speed level) and the degree of strength of voice intonation (hereinafter, referred to as voice intonation level). Therefore, the degree of understandability of the information recipient to the auditory information is improved by lowering the voice speed level (increasing the presentation interval of information or words) or increasing the voice intonation level (strengthening the intonation). Thus, it is possible to suppress the judgment mistakes of the information recipient and reduce the frequency of occurrence judgment mistakes.

Therefore, the information presentation format optimization device 3 of this embodiment determines the attributes of the information recipient based on the output of each sensor 2 when the information presented by the information presenter to the information presenter is auditory information. At the same time, the voice speed level and the voice intonation level of the auditory information are determined, and the appropriateness of the current presentation format of the auditory information to the information recipient is evaluated based on these determination results. Then, the information presentation format optimization device 3 converts the auditory information into a presentation format of the voice speed level and the voice intonation level according to the attributes of the information recipient based on the evaluation result, and outputs the information.

As illustrated in FIG. 1, as a means for realizing the information presentation format optimization function according to this embodiment as described above, the presentation information feature determination program 20, the attribute determination program 21, the feedback optimization program 22, and the prediction model creation program 25 are stored in the memory 11 of the information presentation format optimization device 3, and the determination database group 23 and the optimization database group 24 are stored in the storage device 12 of the information presentation format optimization device 3.

The presentation information feature determination program 20 is a program having a function of detecting a feature of the information necessary for determining the current visual recognition level and cognitive conflict level of the information presented by the information presenter to the information recipient, and the voice speed level and the voice intonation level.

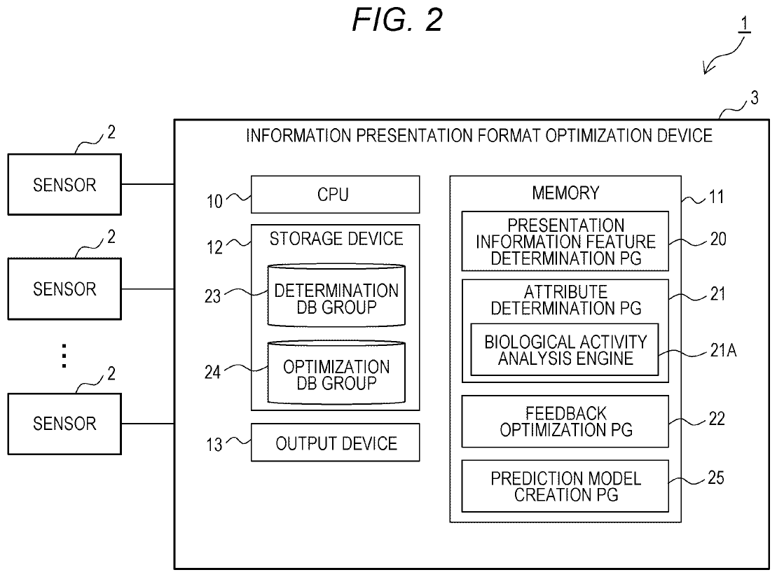

In practice, as illustrated in FIG. 2, the presentation information feature determination program 20 detects, as the “features”, the color combination in the visibility information (hereinafter, referred to a combination of a background color and a character color) and the word character color in the visibility information when the information is visibility information. In addition, the presentation information feature determination program 20 detects, as the “features”, the presence/absence of other languages, the presence/absence of instructions such as two or more continuous processes, tasks, or actions, and the presence/absence of content that the information recipient needs to judge.

Further, the reason why the presence/absence of other languages, the presence/absence of instructions for two or more continuous processes, tasks, or actions, and the presence/absence of content that the information recipient needs to judge are also detected is that these factors also affects the visual recognition level of the visibility information and the cognitive conflict level, when these factors are included, the visual recognition level of the visibility information becomes low, the cognitive conflict level becomes high, and the degree of understandability of the visibility information becomes low.

Therefore, when the information presented by the information presenter to the information recipient is visibility information, the presentation information feature determination program 20 determines the presence/absence of these features while recognizing the content of the visibility information by natural language analysis processing or the like as needed.

Further, when the information is auditory information, the presentation information feature determination program 20 detects the word interval in the auditory information and the intonation strength as the “features”. In addition, the presentation information feature determination program 20 detects, as the “features”, the presence/absence of other languages, the presence/absence of instructions such as two or more continuous processes, tasks, or actions, and the presence/absence of content that the information recipient needs to judge.

Similarly to the case of the visibility information, the reason why the presence/absence of other languages, the presence/absence of instructions for two or more continuous processes, tasks, or actions, and the presence/absence of content that the information recipient needs to judge are also detected is that these factors also affects the degree of understandability of the auditory information, when these factors are included, the degree of understandability of the auditory information becomes low.

Therefore, when the information presented by the information presenter to the information recipient is auditory information, the presentation information feature determination program 20 determines the presence/absence of these features while recognizing the content of the auditory information by voice recognition processing, natural language analysis processing, or the like.

The attribute determination program 21 is a program having a function of determining (estimating) the attributes (knowledge level and understanding level) of the information recipient with respect to the information presented by the information presenter to the information recipient based on the output of each sensor 2. In practice, the attribute determination program 21 includes a biological activity analysis engine 21A (FIG. 1) that acquires a specific feature amount related to the biological activity of the information recipient from the output of each sensor 2. Then, the attribute determination program 21 determines the knowledge level and the understanding level of the information recipient about the information based on each feature amount of the biological activity of the information recipient acquired by the biological activity analysis engine 21A when the information presenter presents the information to the information recipient.

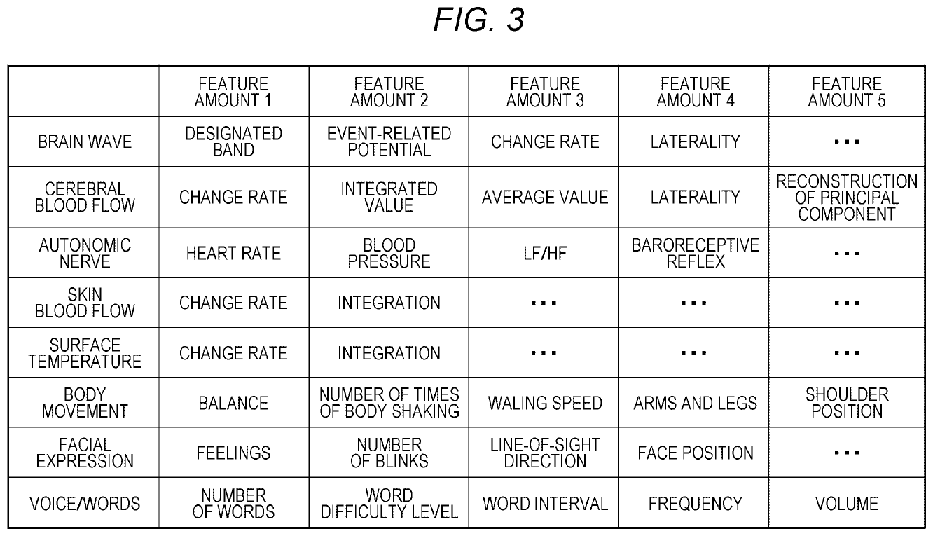

The biological activity analysis engine 21A acquires one or more feature amounts related to biological activity from the output of one sensor 2. For example, as illustrated in FIG. 3, the biological activity analysis engine 21A acquires feature amounts such as brain waves of a designated band (here, 8 to 13 Hz band) of the information recipient, an event-related potential which is a brain reaction due to thinking or cognition, a change rate of brain waves per unit time, and the superiority of left and right brains on the basis of the output of a head gear for measuring brain waves which is one of the sensors 2. For example, if the information recipient has a high knowledge level and a high understanding level, and can fully understand the information presented at that time, the a wave in the 8 to 13 Hz band generated from the brain when relaxing can be observed, so that the knowledge level and the understanding level of the information recipient can be determined based on the brain waves of the predetermined band of the information recipient.

In addition, the biological activity analysis engine 21A acquires the feature amounts such as a change rate of the cerebral blood flow in the frontal lobe of the information recipient per unit time, an integrated value of the change rate, an average value, a latent time, a peak value, laterality, and a reconstructed component of principal component analysis on the basis of the output of a cerebral blood flow measuring device which is one of the sensors 2. It is known that the presented information is processed in the brain, but at this time, if the amount of information is large or complicated judgments are made, the load on the information processing in the brain becomes high, and the cerebral blood flow in the frontal lobe part of the brain becomes high. The knowledge level and the understanding level of the information recipient can be determined based on these feature amounts.

Further, the biological activity analysis engine 21A acquires the feature amounts such as LF/HF and baroreceptive reflex which are indexes of sympathetic nerve calculated by power spectrum analysis on the frequency components of cycle fluctuations of the heart rate, blood pressure, and heart rate of the information recipient based on the output from the sensor 2 such as a heart rate meter, blood pressure meter, and acceleration pulse measuring device that collects information on autonomic nerves. When the knowledge level and understanding level of the information recipient is low and the mental stress for understanding the presented information is large, these numerical values become large, so it is possible to determine the knowledge level and understanding level of the information recipient based on these feature amounts.

Further, the biological activity analysis engine 21A acquires the feature amounts such as the rate of change and integral value of skin blood flow and the rate of change and integral value of the surface temperature of the entire body based on the output of thermography which is one of the sensors 2. When the information recipient has a low knowledge level and understanding level and tense, sympathetic nerve activity is activated and these values increase (however, the surface temperature of the limbs where the peripheral vascular site are present is lowered by the activation of sympathetic nerve activity). Therefore, it is possible to determine the knowledge level and understanding level of the information recipient based on these feature amounts.

Further, the biological activity analysis engine 21A uses a captured image based on the output to acquire, as the feature amounts, physical motions such as balance the information recipient, the number of body shaking per unit time, walking speed, presence/absence of arms and legs, shoulder position, and facial expressions such as feelings, number of blinks per unit time, line-of-sight direction, and face position based on the output (imaging signal) output of the camera, which is one of the sensors 2. In general, when the information recipient has a low knowledge level and understanding level and cannot understand the presented information, physical hand movements such as increasing the number of body shakings per unit time and a facial expression such that the line-of-sight direction faces in a direction different from the information are likely caused. Therefore, it is possible to determine the knowledge level and understanding level of the information recipient based on these feature amounts.

Further, the biological activity analysis engine 21A analyzes the voice based on the output (voice signal) of a microphone, which is one of the sensors 2, to acquire the number of words in the statement of the information recipient, word difficulty level, word interval, frequency, volume, and intonation as feature amounts. When the knowledge level and understanding level of the information recipient is low, the number of words uttered by the information recipient is small and the word difficulty level is low. Therefore, it is possible to determine the knowledge level and understanding level of the information recipient based on these feature amounts.

The feedback optimization program 22 is a program having a function of converting the presentation format of the information presented to the information recipient into a presentation format according to the attributes of the information recipient. In practice, the feedback optimization program 22 determines a presentation format of information considered as optimum to the information recipient based on the characteristics of the information presented to the information recipient determined by the presentation information feature determination program 20 and the knowledge level and understanding level of the information recipient regarding the information which are determined by the attribute determination program 21, and converts the information into the presentation format and presents it to the information recipient. As a result, the information recipient has less misrecognition on the presented content, or easily judge, or has a shorter time to understand.

The prediction model creation program 25 is a program having a function of combining and acquiring information which is acquired from the information recipient by questionnaires or from the evaluation of the subsequent behavior of the information recipient, indicating the effect of converted presentation format (the degree of understanding of the information recipient) stored and held in the storage device 12, and information about knowledge level and understanding level of an information recipient, information presented to the information recipient, and a presentation format converted and presented to the information recipient, sorting the information into learning data and test data, using a method such as regression analysis to create a learning model, and a function of verifying a prediction degree by the learning model using the test data. The prediction model creation program 25 updates the determination database group 23 and the optimization database group 24 (described later) as needed to maximize the effect of the converted presentation format based on the prediction model obtained through such learning, or controls the feedback optimization program 22 to convert the information presented to the information recipient to an optimum presentation format. As a result, it is possible to obtain knowledge about presentation format most suitable for information recipient and the information recipient, and to prepare information based on the presentation format in advance according to the type (beginner, etc.) of information recipient.

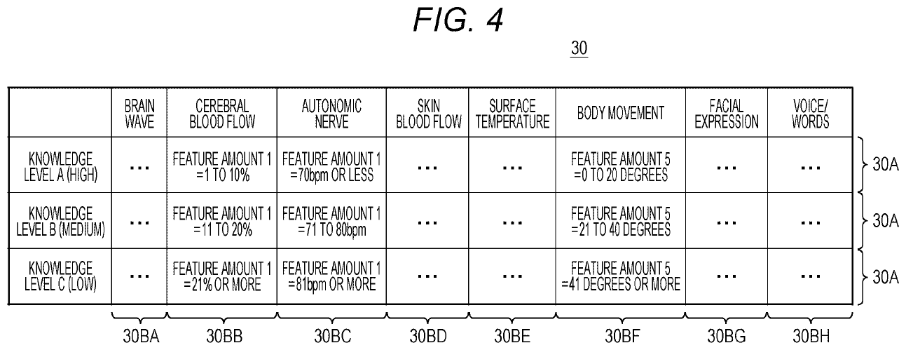

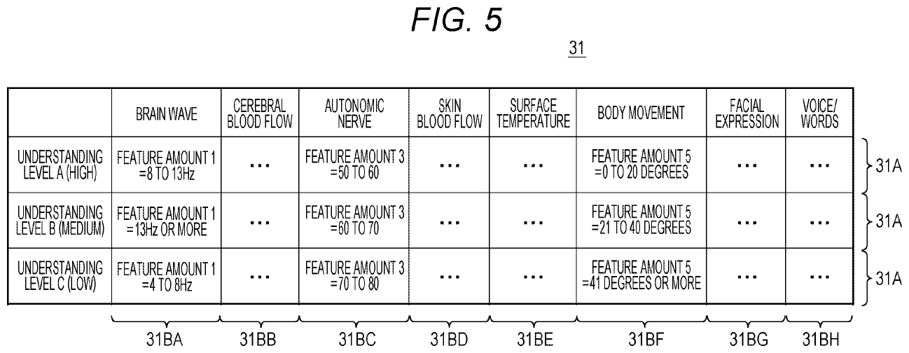

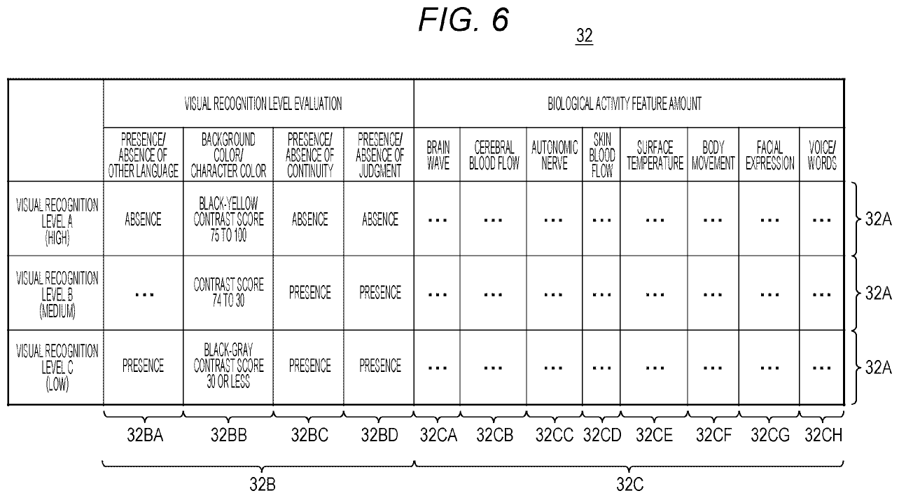

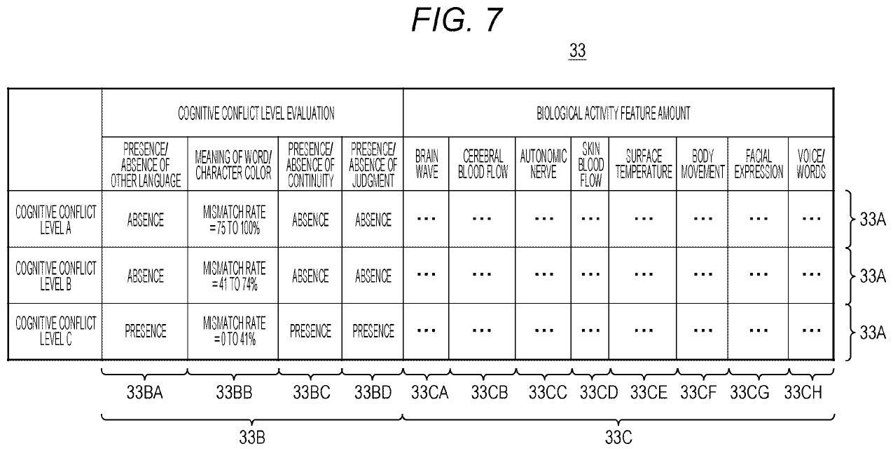

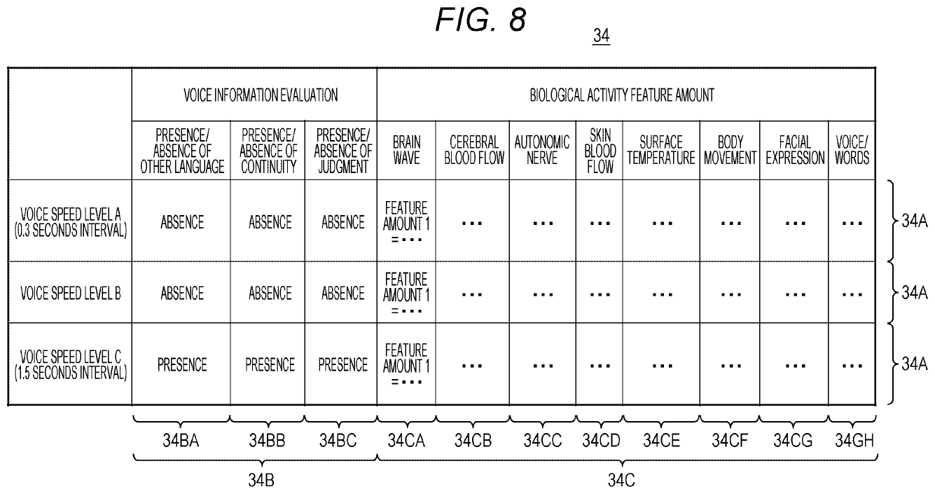

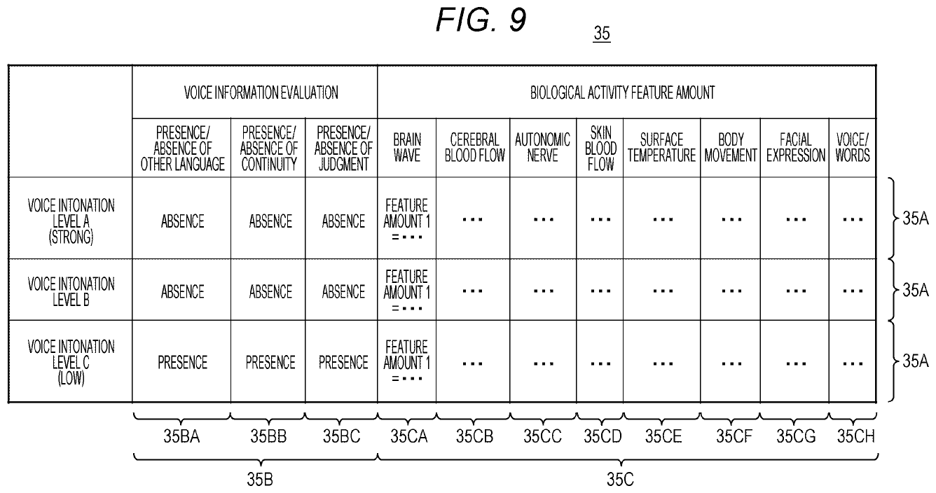

On the other hand, the determination database group 23 includes a knowledge level determination database 30 illustrated in FIG. 4, an understanding level determination database 31 illustrated in FIG. 5, a visual recognition level determination database 32 illustrated in FIG. 6, a cognitive conflict level determination database 33 illustrated in FIG. 7, a voice speed level determination database 34 illustrated in FIG. 8, and a voice intonation level determination database 35 illustrated in FIG. 9.

The knowledge level determination database 30 is a database in which the criteria for each knowledge level when determining (estimating) the knowledge level of the information recipient is stored, and as illustrated in FIG. 4, is configured in a table with three rows 30A associated to three knowledge levels “A” to “C”.

In addition, these rows 30A are divided into a plurality of columns (hereinafter, these will be referred to as feature amount columns) 30BA to 30BH associated to brain waves, cerebral blood flow, autonomic nerves, skin blood flow, surface temperature, body movements, facial expressions, and biological activities such as voice/words which can be acquired by the information presentation format optimization device 3 based on the output from each sensor 2. Then, these feature amount columns 30BA to 30BH each store the value or range of the corresponding feature amount of the corresponding biological activity, which is required to determine the knowledge level of the information recipient as the knowledge level corresponding to the row 30A.

Therefore, in the case of the example of FIG. 4, for example, it is illustrated that it is required to satisfy all the conditions of a feature amount of “1” regarding “cerebral blood flow” (in the example of FIG. 3, “change rate”) is “1 to 10%”, a feature amount of “3” regarding “autonomic nerve” (in the example of FIG. 3, “LF/HF”) is “50 to 60%”, and a feature amount of “5” regarding “body movement” (in the example of FIG. 3, “shoulder position”) is “0 to 20 degrees” in order to determine the knowledge level of the information recipient as “A”.

Further, FIG. 4 illustrates a case where only one criterion for each knowledge level of “A” to “C” is registered in the knowledge level determination database 30, but the actual knowledge level determination database 30 stores a plurality of criteria for each knowledge level. However, only some criteria for each knowledge level of “A” and “C” are registered in the knowledge level determination database 30, and if these criteria are not met, it may be determined that the knowledge level is “B”.

In addition, the understanding level determination database 31 is a database in which the criteria for each understanding level when determining (estimating) the understanding level of the information recipient is stored, and as illustrated in FIG. 5, is configured in a table with three rows 31A associated to three understanding levels “A” to “C”.

In addition, these rows 31A are divided into a plurality of feature amount columns 31BA to 31BH associated to brain waves, cerebral blood flow, autonomic nerves, skin blood flow, surface temperature, body movements, facial expressions, and each biological activity such as voice/words which can be acquired by the information presentation format optimization device 3 based on the output from each sensor 2. Then, these feature amount columns 31BA to 31BH each store the value or range of the feature amount of the corresponding biological activity, which is required to determine the information recipient as having the understanding level corresponding to the row 31A.

Therefore, in the case of the example of FIG. 5, for example, it is illustrated that it is required to satisfy all the conditions of a feature amount of “1” regarding “brain wave” (in the example of FIG. 3, “predetermined band”) is “8 to 13 Hz”, a feature amount of “3” regarding “autonomic nerve” (in the example of FIG. 3, “LF/HF”) is “50 to 60%”, . . . , and a feature amount of “5” regarding “body movement” (in the example of FIG. 3, “shoulder position”) is “0 to 20 degrees”, . . . , in order to determine the understanding level of the information recipient as “A”.

Further, FIG. 5 illustrates a case where only one criterion for each understanding level of “1” to “3” is registered in the understanding level determination database 31, but the actual understanding level determination database 31 stores a plurality of criteria for each understanding level. However, only the criterion for each understanding level of “A” and “C” is registered in the understanding level determination database 31, and if these criteria are not met, it may be determined that the understanding level is “B”.

The visual recognition level determination database 32 is a database in which the criteria for each visual recognition level when determining the visual recognition level of the visibility information presented to information recipient is stored, and as illustrated in FIG. 6, is configured in a table with three rows 32A associated to three visual recognition levels “A” to “C”.

In addition, these rows 32A each are divided into a visual recognition level evaluation column 32B and a biological activity feature amount column 32C, and the color evaluation is further divided into other language presence/absence column 32BA, background color/character color column 32BB, continuity presence/absence column 32BC, and determination presence/absence column 32BD. Then, in these other language presence/absence column 32BA, background color/character color column 32BB, continuity presence/absence column 32BC, and determination presence/absence column 32BD, there is stored a flag indicating the presence/absence of the corresponding “feature” and color combination, which are required for determining the visibility information presented to the information recipient as the visual recognition level of the row 32A.

For example, in the other language presence/absence column 32BA, a flag indicating whether the visibility information presented to the information recipient at that time should include another language is stored. Further, in the background color/character color column 32BB, one of combinations of the character color of the character included in the visibility information and the background color is stored. Further, in the continuity presence/absence column 32BC, a flag indicating whether two or more consecutive work, processing, or operation instructions should be included in the visibility information is stored. In the determination presence/absence column 32BD, a flag indicating whether the visibility information should include a content that requires the judgment of the information recipient is stored.

In addition, the biological activity feature amount column 32C is divided into a brain wave column 32CA, a cerebral blood flow column 32CB, an autonomic nerve column 32CC, a skin blood flow column 32CD, a surface temperature column 32CE, a body movement column 32CF, a facial expression column 32CG, and a voice/word column 32CH. Then, these brain wave column 32CA, cerebral blood flow column 32CB, autonomic nerve column 32CC, skin blood flow column 32CD, surface temperature column 32CE, body movement column 32CF, facial expression column 32CG, and voice/word column 32CH each store the value or range of the feature amount of the biological activity (brain wave, cerebral blood flow, autonomic nerve, skin blood flow, surface temperature, body movement, facial expression, or voice/word) corresponding to the information recipient which is required for determining that the visibility information presented to the information recipient at that time is the visual recognition level of the row 32A.

For example, the value or range of one feature amount related to the brain wave of the information recipient, which is required to determine the visibility information as the visual recognition level of the row 32A, is stored in the brain wave column 32CA. The value or range of one feature amount related to the cerebral blood flow of the information recipient, which is required for determining the visibility information as the visual recognition level of the row 32A is stored in the cerebral blood flow column 32CB.

In addition, the value or range of one feature amount related to the autonomic nerve of the information recipient, which is required to determine the visibility information as the visual recognition level of the row 32A is stored in the autonomic nerve column 32CC. The value or range of one feature amount related to the skin blood flow of the information recipient, which is required for determining the visibility information as the visual recognition level of the row 32A is stored in the skin blood flow column 32CD.

Further, the value or range of one feature amount related to the surface temperature of the information recipient, which is required to determine the visibility information as the visual recognition level of the row 32A, is stored in the surface temperature column 32CE. The value or range of one feature amount related to the body movement of the information recipient, which is required for determining the visibility information as the visual recognition level of the row 32A is stored in the body movement column 32CF.

Further, the value or range of one feature amount related to the facial expression of the information recipient, which is required to determine the visibility information as the visual recognition level of the row 32A, is stored in the facial expression column 32CG. The value or range of one feature amount related to the voice/word of the information recipient, which is required for determining the visibility information as the visual recognition level of the row 32A is stored in the voice/word column 32CH.

Therefore, in the case of the example of FIG. 6, for example, in order to determine the visual recognition level of the visibility information as “A”, it is illustrated that the feature amount of the biological activity of the information recipient is the value or range defined in the visual recognition level determination database 32 on an assumption that yellow characters are displayed in the black background, and the contrast score is “75-100” without including other language, continuous work, an instruction of processing or operation, and judgment of the information recipient are not included in the visibility information (“presence/absence of other language”, “presence/absence of continuity” and “presence/absence of judgment” are all “None”).

Further, FIG. 6 illustrates a case where only one criterion for each visual recognition level of “1” to “3” is registered in the visual recognition level determination database 32, but the actual visual recognition level determination database 32 stores a plurality of criteria for each visual recognition level. However, only the criterion for each visual recognition level of “A” and “C” is registered in the visual recognition level determination database 32, and if these criteria are not met, it may be determined that the visual recognition level of the visibility information is “B”.

The cognitive conflict level determination database 33 is a database in which the criteria for each cognitive conflict level when determining the cognitive conflict level of the visibility information presented to information recipient is stored, and as illustrated in FIG. 7, is configured in a table with three rows 33A associated to three cognitive conflict levels “A” to “C”.

In addition, these rows 33A each are divided into a cognitive conflict level evaluation column 33B and a biological activity feature amount column 33C. The cognitive conflict level evaluation column 33B is further divided into other language presence/absence column 33BA, word character color column 33BB, continuity presence/absence column 33BC, and determination presence/absence column 33BD. The biological activity feature amount column 33C is further divided into brain wave column 33CA, cerebral blood flow column 33CB, autonomic nerve column 33CC, skin blood flow column 33CD, surface temperature column 33CE, body movement column 33CF, facial expression column 33CG, and voice/word column 33CH.

Then, the other language presence/absence column 33BA, the word character color column 33BB, the continuity presence/absence column 33BC, and the determination presence/absence column 33BD of the cognitive conflict level evaluation column 33B each store a flag indicating the presence/absence of the corresponding “feature” (the presence/absence of other language, a mismatch rate of word character color in the auditory information, the presence/absence of an instruction of two or more consecutive works, processing, or operations, and the presence/absence of judgment of the information recipient) and a numerical value indicating the range of a mismatch rate, which are required for determining that the visibility information presented to the information recipient is at the cognitive conflict level of the row 33A.

In addition, the brain wave column 33CA, the cerebral blood flow column 33CB, the autonomic nerve column 33CC, the skin blood flow column 33CD, the surface temperature column 33CE, the body movement column 33CF, the facial expression column 33CG, and the voice/word column 33CH of the biological activity feature amount column 33C each store the value or range of the feature amount corresponding to the biological activity (brain wave, cerebral blood flow, autonomic nerve, skin blood flow, surface temperature, body movement, facial expression, or voice/word) which is required for determining that the visibility information is at the cognitive conflict level of the row 33A.

Therefore, in the case of the example of FIG. 7, for example, in order to determine the cognitive conflict level of the visibility information presented to the information recipient as “A”, it is illustrated that the feature amount of the biological activity of the information recipient is the value or range defined in the cognitive conflict level determination database 33 on an assumption that the mismatch rate between the meaning of each word in the visibility information and the character color is “75-100%” without including other language, an instruction of two or more consecutive works, processing, or operations, and judgment of the information recipient are not included in the visibility information (“presence/absence of other language”, “presence/absence of continuity” and “presence/absence of judgment” are all “None”).

Further, FIG. 7 illustrates a case where only one criterion for each cognitive conflict level of “1” to “3” is registered in the cognitive conflict level determination database 33, but the actual cognitive conflict level determination database 33 stores a plurality of criteria for each cognitive conflict level. However, only the criterion for each cognitive conflict level of “A” and “C” is registered in the cognitive conflict level determination database 33, and if these criteria are not met, it may be determined that the cognitive conflict level of the visibility information is “B”.

The voice speed level determination database 34 is a database in which the criteria for each voice speed level when determining the voice speed level of the auditory information presented to information recipient is stored, and as illustrated in FIG. 8, is configured in a table with three rows 34A associated to three voice speed levels “A” to “C”.

In addition, these rows 34A each are divided into a voice information evaluation column 34B and a biological activity feature amount column 34C. The voice information evaluation column 34B is further divided into other language presence/absence column 34BA, continuity presence/absence column 34BB, and determination presence/absence column 34BC. The biological activity feature amount column 34C is further divided into brain wave column 34CA, cerebral blood flow column 34CB, autonomic nerve column 34CC, skin blood flow column 34CD, surface temperature column 34CE, body movement column 34CF, facial expression column 34CG, and voice/word column 34CH.

The other language presence/absence column 34BA, the continuity presence/absence column 34BB, and the determination presence/absence column 34BC of the voice information evaluation column 34B each store a flag indicating the presence/absence of “feature” corresponding to the auditory information presented from the information presenter which is required for determining that the auditory information presented to the information recipient at that time is at the voice speed level of the row 34A on an assumption that the word interval for “A” of the voice speed level is less than 0.3, and the word interval for “C” of the voice speed level is 1.5 or more.

In addition, the brain wave column 34CA, the cerebral blood flow column 34CB, the autonomic nerve column 34CC, the skin blood flow column 34CD, the surface temperature column 34CE, the body movement column 34CF, the facial expression column 34CG, and the voice/word column 34CH of the biological activity feature amount column 34C each store the value or range of the feature amount of the biological activity (brain wave, cerebral blood flow, autonomic nerve, skin blood flow, surface temperature, body movement, facial expression, or voice/word) corresponding to the information recipient which is required for determining that the auditory information is at the voice speed level of the row 34A.

Therefore, in the case of the example of FIG. 8, for example, in order to determine the voice speed level of the auditory information as “A”, it is illustrated that the feature amount of the biological activity of the information recipient is the value or range defined in the voice speed level determination database 34 on an assumption that the intervals of information and words is less than “0.3 seconds” without including other language, an instruction of two or more consecutive works, and judgment of the information recipient are not included in the visibility information (“presence/absence of other language”, “presence/absence of continuity” and “presence/absence of judgment” are all “None”).

Further, FIG. 8 illustrates a case where only one criterion for each voice speed level of “1” to “3” is registered in the voice speed level determination database 34, but the actual voice speed level determination database 34 stores a plurality of criteria for each knowledge level. However, only the criterion for each voice speed level of “A” and “C” is registered in the voice speed level determination database 34, and if these criteria are not met, it may be determined that the voice speed level of the auditory information is “B”.

The voice intonation level determination database 35 is a database in which the criteria for each voice speed level when determining the voice intonation level of the auditory information presented to information recipient is stored, and as illustrated in FIG. 9, is configured in a table with three rows 35A associated to three voice intonation levels “A” to “C”.

In addition, these rows 35A each are divided into a voice information evaluation column 35B and a biological activity feature amount column 35C. The voice information evaluation column 35B is further divided into other language presence/absence column 35BA, continuity presence/absence column 35BB, and determination presence/absence column 35BC. The biological activity feature amount column 35C is further divided into brain wave column 35CA, cerebral blood flow column 35CB, autonomic nerve column 35CC, skin blood flow column 35CD, surface temperature column 35CE, body movement column 35CF, facial expression column 35CG, and voice/word column 35CH.

The other language presence/absence column 35BA, the continuity presence/absence column 35BB, and the determination presence/absence column 35BC of the voice information evaluation column 35B each store a flag indicating the presence/absence of “feature” (the presence/absence of other language, the presence/absence of an instruction of two or more consecutive works, processing, or operations, and the presence/absence of judgment of the information recipient) corresponding to the auditory information which is required for determining that the auditory information presented to the information recipient is the voice intonation level of the row 35A on an assumption that the intonation for “A” of the voice intonation level is stronger than a predetermined first threshold and the intonation for “C” of the voice intonation level is lower than a predetermined second threshold which is smaller than the first threshold.

In addition, the brain wave column 35CA, cerebral blood flow column 35CB, autonomic nerve column 35CC, skin blood flow column 35CD, surface temperature column 35CE, body movement column 35CF, facial expression column 35CG, and voice/word column 35CH of the biological activity feature amount column 35C each store the value or range of the feature amount of the biological activity (brain wave, cerebral blood flow, autonomic nerve, skin blood flow, surface temperature, body movement, facial expression, or voice/word) corresponding to the information recipient which his required for determining that the auditory information is the voice intonation level of the row 35A.

Therefore, in the case of the example of FIG. 9, for example, in order to determine the voice intonation level of the auditory information as “A”, it is illustrated that the feature amount of the biological activity of the information recipient is the value or range defined in the voice intonation level determination database 35 on an assumption that the intonation is strong without including other language, an instruction of two or more consecutive works, processing, or operations, and judgment of the information recipient are not included in the auditory information (“presence/absence of other language”, “presence/absence of continuity” and “presence/absence of judgment” are all “None”).

Further, FIG. 9 illustrates a case where only one criterion for each voice intonation level of “1” to “3” is registered in the voice intonation level determination database 35, but the actual voice intonation level determination database 35 stores a plurality of criteria for each voice intonation level. However, only the criterion for each voice intonation level of “A” and “C” is registered in the voice intonation level determination database 35, and if these criteria are not met, it may be determined that the voice intonation level of the auditory information is “B”.

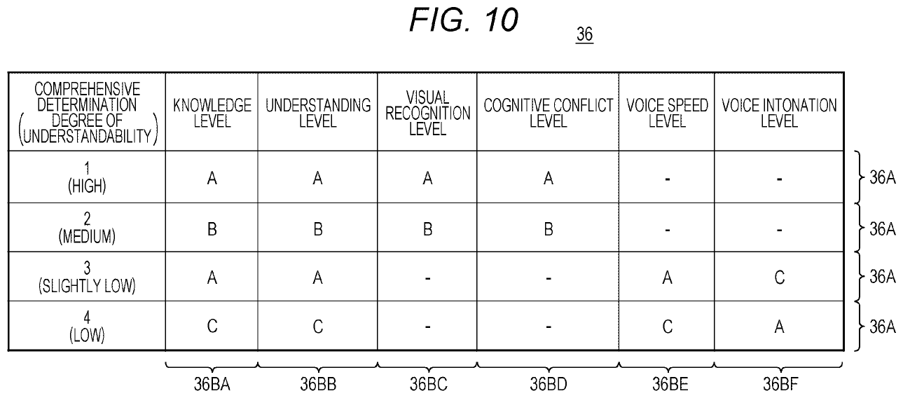

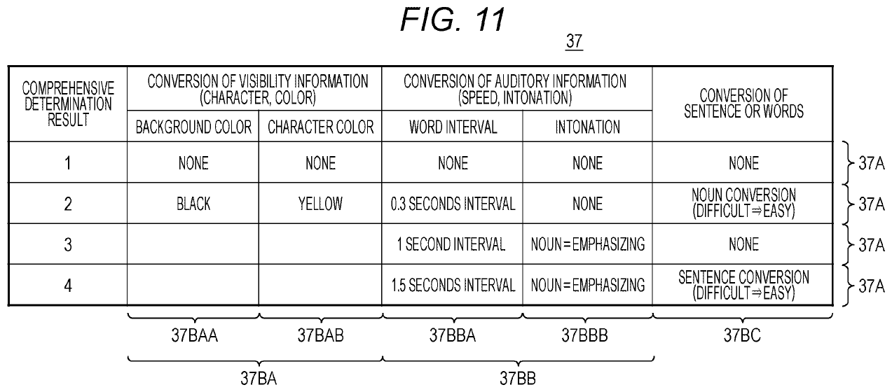

On the other hand, the optimization database group 24 (FIG. 1) is configured by a comprehensive determining optimization database 36 illustrated in FIG. 10 and a conversion optimization database 37 illustrated in FIG. 11.

The comprehensive determining optimization database 36 is a database where the degree of understandability information of the information recipient to the visibility information or the auditory is comprehensively determined based on the knowledge level and the understanding level of the information recipient determined as described above, the visual recognition level and the cognitive conflict level of the visibility information presented to the information recipient, or the voice speed level and the voice intonation level of the auditory information presented to the information recipient.

As illustrated in FIG. 10, the comprehensive determining optimization database 36 is configured in a table shape having rows 36A associated to each of the four degrees of understandability “1” to “4”. In addition, these rows 36A each are divided into a knowledge level column 38BA, an understanding level column 36BB, a visual recognition level column 36BC, a cognitive conflict level column 36BD, a voice speed level column 36BE, and a voice intonation level column 36BF.

The knowledge level column 38BA, the understanding level column 36BB, the visual recognition level column 36BC, the cognitive conflict level column 36BD, the voice speed level column 36BE, and the voice intonation level column 36BF each store the knowledge level, the understanding level, the visual recognition level, the cognitive conflict level, the voice speed level, or the voice intonation which is required for determining that the current presentation format of information presented to the information recipient is the degree of understandability corresponding to the row 36A depending on the information recipient.

Therefore, in the case of the example of FIG. 10, for example, when the knowledge level and the understanding level of the information recipient are both “A”, and the visual recognition level and the cognitive conflict level of the information presented to the information presenter are both “A”, it is indicated that the degree of understandability of the current presentation format of the information should be determined to be “1” for the information recipient.

In the example of FIG. 10, the combination of the knowledge level, the understanding level, the visual recognition level, the cognitive conflict level, the voice speed level, and the voice intonation level at which the comprehensive judgment (degree of understandability) is “1” to “4” is stored by only one in the comprehensive determining optimization database 36. However, in practice, all combinations of the knowledge level, the understanding level, the visual recognition level, and the cognitive conflict level, and all combinations of the knowledge level, the understanding level, and the voice speed level, and the voice intonation level are sorted into any of “1” to “4” of the comprehensive determination, and stored in the comprehensive determining optimization database 36.

The conversion optimization database 37 is a database that stores the converted presentation format of the corresponding information, which is preset for each of the degree of understandability of “1” to “4”.

As illustrated in FIG. 11, the conversion optimization database 37 is configured in a table shape having rows 37A associated to each of the four degrees of understandability “1” to “4”. In addition, these rows 37A each are divided into a visibility information conversion method column 37BA, an auditory information conversion method column 37BB, and a sentence/word conversion method column 37BC. The visibility information conversion method column 37BA is further divided into a background color column 37BAA and a character color column 37BAB. The auditory information conversion method column 37BB is further divided into a voice speed column 37BBA and an intonation column 37BBB.

The background color column 37BAA and the character color column 37BAB of the visibility information conversion method column 37BA store a background color or a character color to be applied when the visibility information is presented to the information recipient in a case where the information presented to the information recipient is the visibility information, and a comprehensive determination result of the visibility information is the degree of understandability of the corresponding row 37A.

In addition, the voice speed column 37BBA and the intonation column 37BBB of the auditory information conversion method column 37BB each store the interval of information or words and the intonation method to be applied when the auditory information is presented to the information recipient in a case where the information presented to the information recipient is auditory information, and the comprehensive determination result of the auditory information is the degree of understandability of the corresponding row 37A.

Further, the sentence/word conversion method column 37BC stores a conversion method of a sentence or words to be applied when the information is presented to the information recipient in a case where the comprehensive determination result of the information (visibility information and auditory information) presented to the information recipient is the degree of understandability of the corresponding row 37A.

Therefore, in the case of the example of FIG. 11, for example, when the degree of understandability of the information presented to the information recipient is “1”, it is illustrated that the information is not converted in any way (all “none”), but the information should be presented to the recipient as it is. Further, in FIG. 11, It is illustrated that, when the degree of understandability of the information presented to the information recipient is “2”, the background color is converted to “black” and the character color is converted to “yellow” when the information is visibility information, and the noun contained in the information should be converted into a more understandable noun and presented to the information recipient.

### (3) Various Processes Related to the Information Presentation Format Optimization Function

Next, the processing contents of various processes executed in the information presentation format optimization device 3 in relation to the above-mentioned information presentation format optimization function will be described. In the following, the subject of various processes will be described as a “program”, but in practice, it is needless to say that the CPU 10 (FIG. 1) of the information presentation format optimization device 3 executes the processes based on the “program”.

### (3-1) Information Presentation Format Optimization Process

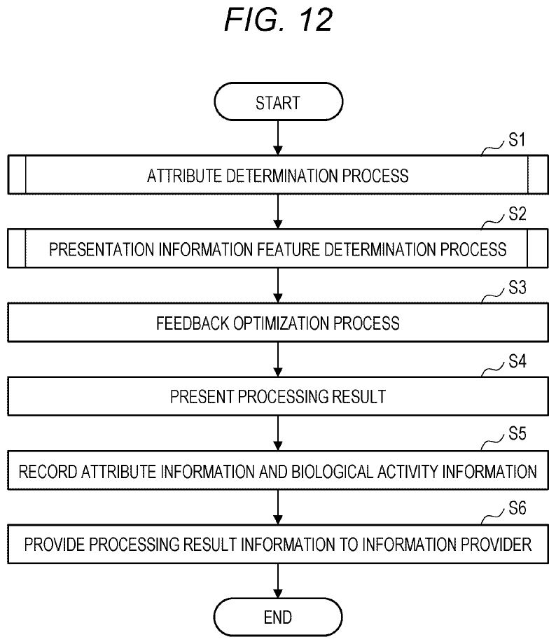

FIG. 12 illustrates a flow of a series of processes executed in the information presentation format optimization device 3 in relation to the above-mentioned information presentation format optimization function. An information presentation method optimization device 1 converts the information presented to the information recipient into a presentation format according to the attributes of the information recipient according to the processing procedure illustrated in FIG. 12.

In practice, in the information presentation format optimization device 3, when the information presenter starts presenting the information to the information recipient, the information presentation format optimization process illustrated in FIG. 12 is started. First, the attribute determination program 21 performs an attribute determination process for determining the attribute of the information recipient based on an electric signal given from each sensor 2 at that time (S1). Then, the attribute determination program 21 then calls the presentation information feature determination program 20.

When the presentation information feature determination program 20 is called by the attribute determination program 21, a presentation information feature determination process is performed for determining the characteristics of the information presented to the information recipient at that time (S2). Then, the presentation information feature determination program 20 then calls the feedback optimization program 22.

When being called by the presentation information feature determination program 20, the feedback optimization program 22 converts the display format of the information presented to the information recipient at that time to a presentation format according to the attribute (knowledge level and understanding level) of the information recipient based on the attribute of the information recipient with respect to the information determined in Step S1, the visual recognition level and the cognitive conflict level of the information determined in Step S2 (a case where the information is visibility information), or voice speed level and voice intonation level (a case where the information is auditory information) (S3). Further, the feedback optimization program 22 presents the information after the presentation format is converted to the information recipient via the output device 13 (FIG. 1) (S4).

Subsequently, the feedback optimization program 22 stores the attribute of the information recipient determined in Step S1, various feature amounts related to the biological activity of the information recipient obtained at that time, and information after the presentation format conversion in the storage device 12 (FIG. 1) (S5). Further, the feedback optimization program 22 presents the attribute of the information recipient stored in the storage device 12 in FIG. 5 and the information after the presentation format conversion to the information presenter (S6), and then ends the process. As described above, this information presentation format optimization process ends.

### (3-2) Attribute Determination Process

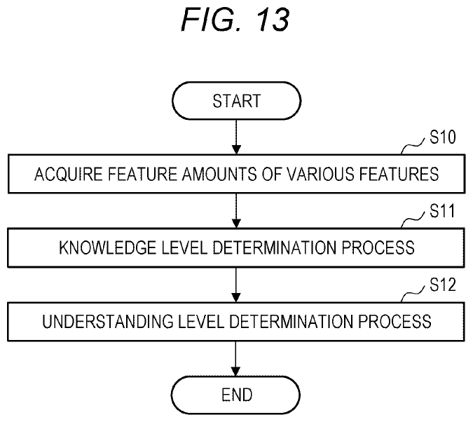

FIG. 13 illustrates a flow of the attribute determination process executed by the attribute determination program 21 in Step S1 of the information presentation format optimization process described above for FIG. 12. When the attribute determination program 21 proceeds to Step S1 of the information presentation format optimization process, the attribute determination process illustrated in FIG. 13 is started. First, based on the output of each sensor 2, each feature amount of the biological activity of the information recipient described in FIG. 3 is acquired (S10).

Subsequently, the attribute determination program 21 refers to the knowledge level determination database 30 (FIG. 4) based on each feature amount acquired in Step S10, and determines the knowledge level of the information recipient for the information presented at that time (S11). In addition, the attribute determination program 21 refers to the understanding level determination database 31 (FIG. 5) based on each feature amount acquired in Step S10, and determines the understanding level of the information recipient at that time for the information (S12).

Then, the attribute determination program 21 then calls the presentation information feature determination program 20 and ends the attribute determination process.

### (3-3) Presentation Information Feature Determination Process

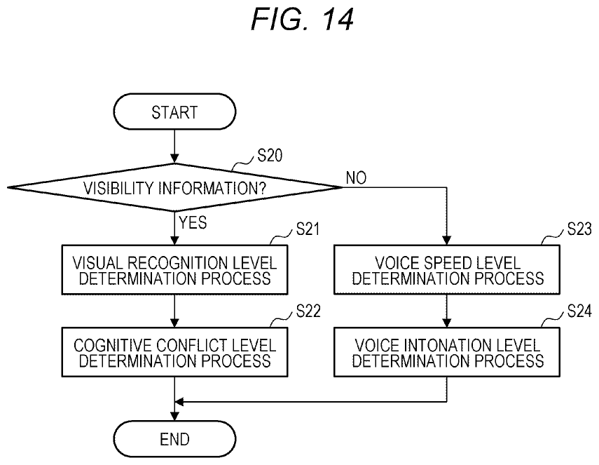

On the other hand, FIG. 14 illustrates a flow of the presentation information feature determination process executed by the presentation information feature determination program 20 in Step S2 of the information presentation format optimization process described above for FIG. 12.

When the presentation information feature determination program 20 is called by the attribute determination program 21, the presentation information feature determination process illustrated in FIG. 14 is started, and it is determined whether the information presented to the information recipient at that time is visibility information (S20).

Then, when the presentation information feature determination program 20 obtains an affirmative result in this determination, the presentation information feature determination program 20 determines the visual recognition level of the information by using the visual recognition level determination database 32 (FIG. 6) (S21). Further, the presentation information feature determination program 20 determines the cognitive conflict level of the information using the cognitive conflict level determination database 33 (FIG. 7) (S22), and then ends the presentation information feature determination process.

On the other hand, when the presentation information feature determination program 20 obtains a negative result in the determination in Step S20, the presentation information feature determination program 20 determines the voice speed level of the information by using the voice speed level determination database 34 (FIG. 8) (S23). Further, the presentation information feature determination program 20 determines the speech intonation level of the information using the voice intonation level determination database 35 (S24), and then ends the presentation information feature determination process.

### (3-4) Learning Process

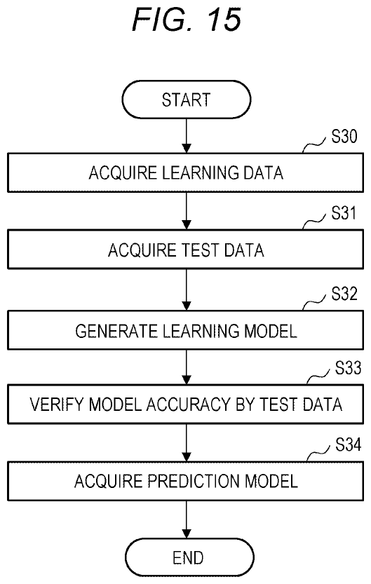

On the other hand, FIG. 15 illustrates a flow of the learning process that is periodically performed by the prediction model creation program 25. The prediction model creation program 25 updates the determination database group 23 and the optimization database group 24 according to the processing procedure illustrated in FIG. 15, and generates a prediction model used to convert the information presented to the information recipient to an optimum presentation format.

In practice, when the prediction model creation program 25 starts the learning process illustrated in FIG. 15, first, the data before and after the conversion of the information whose presentation format has been converted in the past, which is stored in the storage device 12 (FIG. 1), and the effect of the presentation format conversion acquired from the information recipient by questionnaires or from the evaluation of the information recipient's subsequent behavior are read from the storage device 12 (FIG. 1) (S30).

Subsequently, the prediction model creation program 25 acquires the data before and after the conversion of the information whose presentation format has been converted in the past and the data based on the effect of the conversion, for example, 80% of the total data as learning data (S30), and acquires 20% as test data (S31). After that, learning that maximizes the effect is performed by various methods such as machine learning, deep learning, neural network, and genetic algorithm, and a learning model is generated (S32).

Then, the prediction model creation program 25 verifies the model accuracy of predicting the effect of the presentation format conversion on the learning model generated in Step S32 using the test data (S33). The prediction model with the highest effect is selected (S34), and then this learning process is terminated.

After that, the prediction model creation program 25 updates the determination database group 23 and the optimization database group 24 using the prediction model selected in Step S34, and controls the feedback optimization program 22 to convert the information presented to the information recipient to an optimum presentation format.

### (4) Effect of this Embodiment

As described above, the information presentation format optimization device 3 of this embodiment acquires the feature of the information presented by the information presenter to the information recipient, and determines the attribute (knowledge level and understanding level) of the information of the information recipient based on the biological activity information of the information recipient, and determines the degree of understandability of the information for the information recipient based on the feature of the information and the attribute of the information recipient. Then, based on this determination result, the information presentation format optimization device 3 converts the presentation format of the information into a presentation format according to the attribute of the information recipient and presents the information to the information presenter.

Therefore, according to the information presentation format optimization device 3, the information can be presented to the information recipient in the optimum presentation format that the information recipient can easily understand, so that it is possible to prevent misrecognition and lack of understanding of the information recipient about the information beforehand and effectively. In addition, the time required for understanding and judgment can be shortened.

Further, in the information presentation format optimization device 3, since the attribute of the information recipient and the information after the presentation format is converted are presented to the information presenter, the information presenter can refer to the presentation format of the information when the information is presented to an information recipient having the same level of the information recipient next time, for example, at the time of training for new employees. As a result, the information provider can prepare the information format to be given to the information recipient in advance, and can maximize the understanding of the information recipient.

### (5) Other Embodiments

In the above-described embodiment, only the biological activity information of the information recipient has been acquired, and the knowledge level and understanding level of the information recipient have been determined (estimated) based on the acquired biological activity information, but the invention is not limited to this. The biological activity information of the information presenter may also be acquired by sensing using a sensor, and the knowledge level and understanding level of the information presenter may be determined (estimated) based on the acquired biological activity information, and both of these pieces of information may be presented to the information presenter. As a result, it is possible to know whether the knowledge level and understanding level of each other match, and it is possible to shorten the time for exchanging information between the two parties.

Further, in the above-described embodiment, the case where the knowledge level and understanding level of the information recipient for the information are determined based on the brain wave, the cerebral blood flow, and the like has been described, but the invention is not limited to this. A microphone may be provided as the sensor 2, and the content of the information spoken by the information recipient may be collected by the microphone and recognized by voice recognition. Then, the knowledge level and the understanding level of the information recipient may be determined based on the information such as the number of recognized words and the number of recognized technical terms used by the information recipient.

Further, in the above-described embodiment, the case where the above-mentioned various feature amounts are applied to FIG. 3 as the feature amounts of the biological activity used when determining the knowledge level and the understanding level of the information recipient has been described, but the invention is not limited to this. Various other features of biological activity can be widely applied.

Further, in the above-described embodiment, the case where the invention is applied to the information processing system 1 used when a senior worker educates a plurality of new workers about work procedures and the like in a factory has been described, but the invention is not limited to this. The invention can be widely applied in various other situations such as rehabilitation support, general information presentation services for presenting information to customers, remote business support systems for medical care, education, learning, conferences, factories, and the like.

The invention can be widely applied to various information processing systems that optimize the presentation format of information presented by the information presenter to the information recipient.

What is claimed is:

1. An information processing device for optimizing information presented by an information presenter to an information recipient, comprising:
an attribute determination unit that determines a knowledge level and an understanding level of the information recipient with respect to the information based on biological activity information of the information recipient acquired by a sensor;
a presentation information feature determination unit that determines a feature of the information presented by the information presenter to the information recipient; and
a feedback optimization unit that converts a presentation format of the information into a presentation format according to the knowledge level and the understanding level of the information recipient based on the knowledge level and the understanding level of the information recipient with respect to the information determined by the attribute determination unit and the feature of the information determined by the presentation information feature determination unit.

2. The information processing device according to claim 1, wherein
the presentation information feature determination unit determines, as the feature of the information presented by the information presenter to the information recipient,
a visual recognition level which is a degree at which the information can be visually recognized at a glance, and a cognitive conflict level which is a degree of cognitive conflict of the information recipient with respect to the intonation in a case where the information is visual information, and
a voice speed level which is a degree of word interval in the information, and a voice intonation level which is a degree of strength of voice intonation in a case where the information is auditory information.

3. The information processing device according to claim 2, wherein the presentation information feature determination unit determines
the visual recognition level based on a presence/absence of other language in the information, a combination of a background color and a character color, a presence/absence of continuous information, and a presence/absence of judgment of the information recipient, and
the cognitive conflict level based on a presence/absence of other language in the information, a meaning of words and a character color of the words, a presence/absence of continuous information, and a presence/absence of judgment of the information recipient.

4. The information processing device according to claim 2, wherein the presentation information feature determination unit determines
the voice speed level based on an interval of the words, a presence/absence of other language in the information, a presence/absence of continuous information, and a presence/absence of judgment of the information recipient, and
the voice intonation level based on a strength of intonation, a presence/absence of other language in the information, a presence/absence of continuous information, and a presence/absence of judgment of the information recipient.

5. The information processing device according to claim 1, wherein the knowledge level and the understanding level of the information recipient with respect to the information determined by the attribute determination unit, and the information after converting a presentation format according to the knowledge level and the understanding level of the information recipient by the feedback optimization unit are presented to the information presenter.

6. An information processing method for optimizing information presented by an information presenter to an information recipient using an information processing device, comprising:
a first step in which a knowledge level and an understanding level of the information recipient with respect to the information are determined based on a biological activity of the information recipient acquired by a sensor, and a feature of the information presented by the information presenter to the information recipient is determined; and
a second step in which a presentation format of the information is converted into a presentation format according to the knowledge level and the understanding level of the information recipient based on the determined knowledge level and the determined understanding level of the information recipient with respect to the information and the feature of the information.

7. The information processing method according to claim 6, wherein, in the first step,
as the feature of the information presented by the information presenter to the information recipient,
a visual recognition level which is a degree at which the information can be visually recognized at a glance, and a cognitive conflict level which is a degree of cognitive conflict of the information recipient with respect to the intonation are determined in a case where the information is visual information, and
a voice speed level which is a degree of word interval in the information, and a voice intonation level which is a degree of strength of voice intonation are determined in a case where the information is auditory information.

8. The information processing method according to claim 6, wherein, in the first step,
the visual recognition level is determined based on a presence/absence of other language in the information, a combination of a background color and a character color, a presence/absence of continuous information, and a presence/absence of judgment of the information recipient, and
the cognitive conflict level is determined based on a presence/absence of other language in the information, a meaning of words and a character color of the words, a presence/absence of continuous information, and a presence/absence of judgment of the information recipient.

9. The information processing method according to claim 6, wherein, in the first step,
the voice speed level is determined based on an interval of the words, a presence/absence of other language in the information, a presence/absence of continuous information, and a presence/absence of judgment of the information recipient, and
the voice intonation level is determined based on a strength of intonation, a presence/absence of other language in the information, a presence/absence of continuous information, and a presence/absence of judgment of the information recipient.

10. The information processing method according to claim 6, further comprising:
a third step in which the determined knowledge level and the determined understanding level of the information recipient with respect to the information, and the information after converting a presentation format according to the knowledge level and the understanding level of the information recipient are presented to the information presenter.
